(19) United States
(12) Plant Patent Application Publication
(10) Pub. No.: US 2004/0194181 P1
(43) Pub. Date: Sep. 30, 2004

(54) Torenia plant named SUNRENILAPIHO-KAI

(76) Inventor: Kazunari Iwaki, Omi-hachiman-shi (JP)

Correspondence Address: CHRISTIE, PARKER & HALE, LLP, PO BOX 7068, PASADENA, CA, 91109-7068, US

(21) Appl. No.: 10/405,070

(22) Filed: Mar. 31, 2003

(51) Int. Cl. A01H 5/00

(52) U.S. Cl. PLT/263

(57) ABSTRACT

Disclosed herein is a Torenia plant has large flowers, moderate purplish pink upper petal with vivid reddish purple bilabiate petal without yellow blotches and vivid reddish purple floral tube. The plant has semi-erect growth habit, medium branching and great profusion of blooms. The whole bush remains in bloom for a considerable period of time. The plant grows and has flowers ordinarily in the shade of trees.

### BACKGROUND OF THE VARIETY

The present innovation relates to a new and distinct variety of Torenia plant obtained from artificial doubling of ‘SUNRENIPIHO’ (the Seeds and Seedlings Law Registration No. 7512; U.S. Pat. No. PLANT 10118).

In September 1999, the cuttings of ‘SUNRENIPIHO’ were treated with 0.1% colchicines solution, which were propagated by cutting on the peat at Omi R&D Center, Suntory Flowers Ltd. residing at 863-1, Aza-Iketani, Omori-cho, Youkaichi-shi, Shiga-ken, Japan. After two months, some survived plantlets were transplanted in pots. In December 1998, colchiploids were obtained from the selection. The discovered Torenia plant was propagated by cutting and then grown in bed and pot on trial. The botanical characteristics of the plant were examined using the parent variety ‘SUNRENIPIHO’ and the similar variety ‘SUNRENILAPIHO’ for comparison. As a result, it was concluded that this discovered Torenia plant is distinguishable from any other variety, whose existence is known to us, sufficiently uniform and stable in its characteristics. Then this new variety of Torenia plant was named ‘SUNRENILAPIHO-KAI’.

### SUMMARY OF THE VARIETY

The new variety of torenia plant named ‘SUNRENILAPIHO-KAI’ has large flowers, moderate purplish pink upper petal with vivid reddish purple bilabiate petal without yellow blotches and vivid reddish purple floral tube. The plant has semi-erect growth habit, medium branching and great profusion of blooms. The whole bush remains in bloom for a considerable period of time. The plant grows and has flowers ordinarily in the shade of trees.

### BRIEF DESCRIPTION OF THE DRAWINGS

FIG. 1 is a photograph giving a partial view of the new variety of torenia plant planted in a flowerpot.

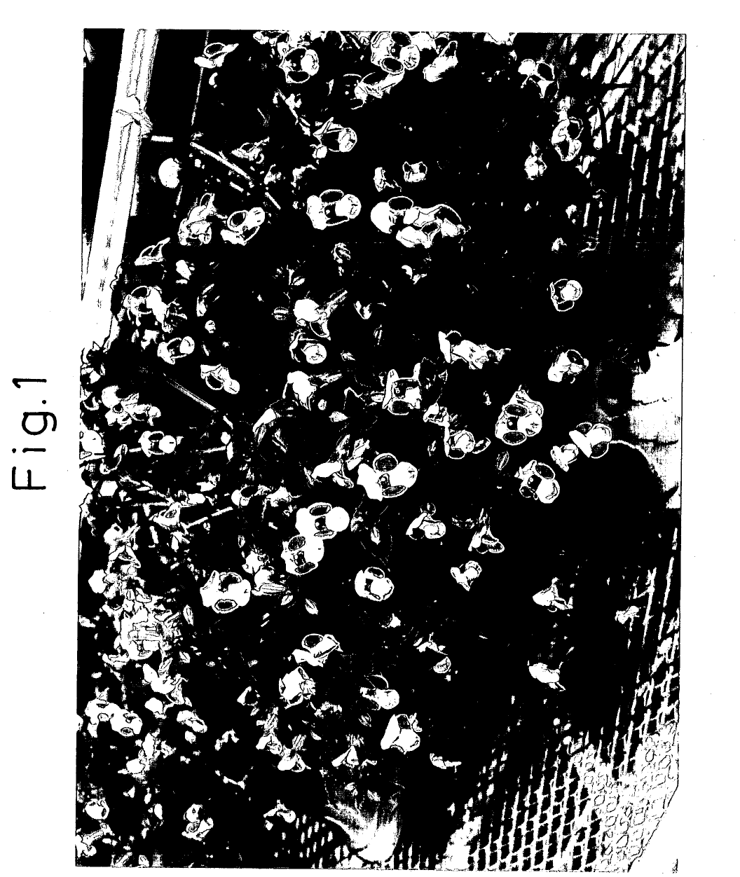

FIG. 2 is a photograph of a flower of the new variety of torenia plant.

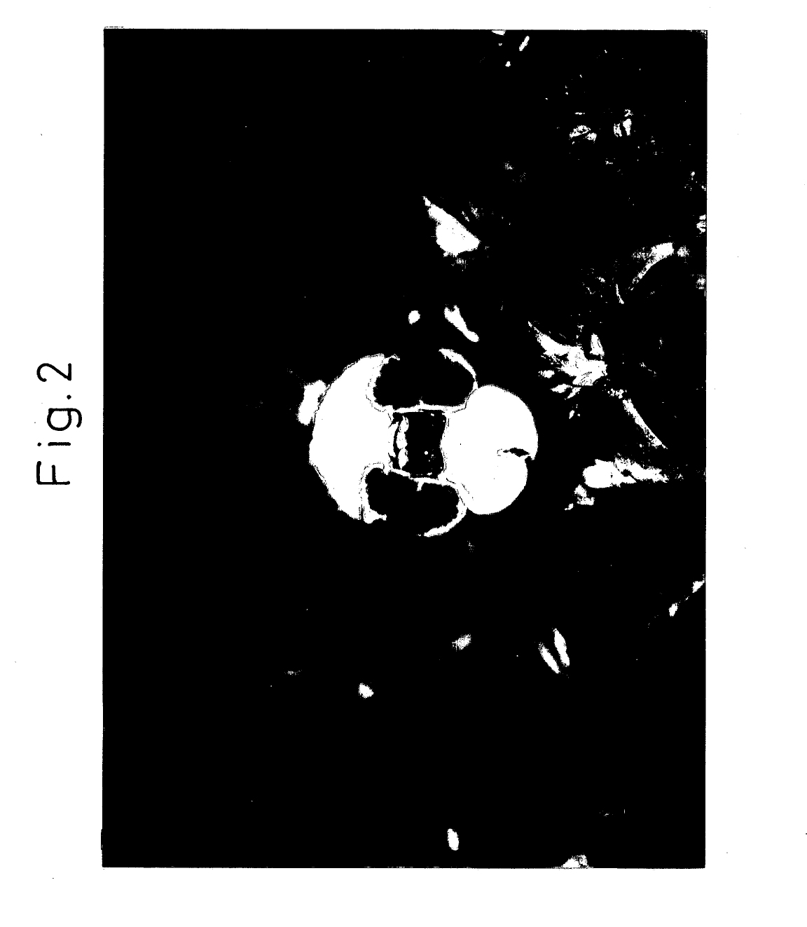

### DESCRIPTION OF THE VARIETY

‘SUNRENILAPIHO-KAI’ has semi-erect growth habit. The plant height is 15 cm with medium branching. Diameter of stem is 2.5 mm. Anthocyanin pigmentation of stem is moderate. Internode length is 5.0 cm. The shape of blade is lanceolate with acute serration. Length of leaf is 3.5 cm and width is 3.0 cm. Pubescence is sparse. Inflorescence is axillary. Diameter of flower is 30 mm and length is 35 mm. The color of upper petal is pale purplish pink (R.H.S. No. 69D, J.H.S. No. 9501). The side of bilabiate petal has vivid purplish red (R.H.S. No. N80A, J.H.S. No. 8906). The bottom of bilabiate petal has vivid purplish red (R.H.S. No. N80A, J.H.S. No. 8906) color and pale purplish pink (R.H.S. No. 69D, J.H.S. No. 9501) center. The color of flower tube is vivid purplish red (R.H.S. No. N80A, J.H.S. No. 8906). Calyx length is 18 mm, and anthocyanin pigmentation of calyx is medium. Peduncle is 2.0 cm in length, 1.5 mm in thickness. Flowering duration is medium. Flowering period is from June to November around Kansai district of Japan.

Plant:

Growth habit.—Semi-erect.

Plant height.—15 cm.

Stem:

Diameter.—2.5 mm.

Anthocyanin pigmentation.—Medium.

Branching.—Medium.

Pubescence.—Sparse.

Length of internode.—5.0 cm.

Leaf:

Shape of blade.—Lanceolate.

Length.—3.5 cm.

Width.—3.0 cm.

Color. (upper side).—R.H.S. 147A.

Pubescence of upper side.—Sparse.

Thickness of petiole.—Medium.(1.5 mm).

Length of petiole.—Short.(7.0 mm).

Flower:

Inflorescence peduncles.—Axillary.

Flower form.—Globose.

Flower length.—Medium to long. (35 mm).

Flower width.—Medium to wide. (30 mm).

Length of tubular.—Medium. (33 mm).

Color of floral tube.—R.H.S. N80A.

Color of petal.—R.H.S. 69D/N80A.

Yellow brotches of the petal.—Absent.

Vertical line of the petal.—Present.

Wave of upper bilabiate petal.—Strong.

Calyx shape.—Shallowly five lobed.

Calyx;Degree of development of wings.—Medium.

Calyx length.—Medium. (18 mm).

Anthocyanin pigmentation of calyx limb.—Medium.

Anthocyanin pigmentation of anther.—Light.

Anther spur.—Absent.

Peduncle thickness.—Thick. (1.5 mm).

Peduncle length.—Short. (20 mm).

Spike existence.—Absent.

Spike; number of flower.—Few.

Flowering duration.—Medium.

Physiological and ecological characteristics: Medium resistance to diseases and pests, strong tolerance to heat, and medium tolerance to cold.

This new variety ‘SUNRENILAPIHO-KAI’ of torenia plant is most suitable for flower bedding and potting, particularly in hanging pots or planters.

This new and distinct variety of Torenia plant, ‘SUNRENILAPIHO-KAI’, was asexually reproduced by cutting at aforementioned Omi R&D Center, Suntory Flowers Ltd., and the homogeneity and stability thereof were confirmed.

### COMPARISON WITH SIMILAR VARIETY

The new variety of torenia plant ‘SUNRENILAPIHO-KAI’ is differ from a parent variety, ‘SUNRENIPIHO’ in following points,

1. The petal color of ‘SUNRENILAPIHO-KAI’ is pale purplish pink (R.H.S. No. 69D, J.H.S. No. 9501) in upper petal, and vivid purplish red (R.H.S. No. 80A, J.H.S. No. 8906) in bilabiate petal as against those of ‘SUNRENIPIHO’ is purplish white (R.H.S. No. 76D, J.H.S. No. 9201), and vivid purple (R.H.S. No. 81A, J.H.S. No. 8606).

2. The flower of ‘SUNRENILAPIHO-KAI’ is larger than that of ‘SUNRENIPIHO’.

3. The peduncle length of ‘SUNRENILAPIHO-KAI’ is shorter than that of ‘SUNRENIPIHO’.

4. The diameter of flower of ‘SUNRENILAPIHO-KAI’ is wider than that of ‘SUNRENIPIHO’.

5. The stem diameter of ‘SUNRENILAPIHO-KAI’ is wider than that of ‘SUNRENIPIHO’.

6. The anthocyanin pigmentation of stem of

‘SUNRENILAPIHO-KAI’ are medium as against these of ‘SUNRENIPIHO’ are dark.

7. The degree of leaf serration of ‘SUNRENILAPIHO-KAI’ is acute as against these of ‘SUNRENIPIHO’ is medium.

The new variety of Torenia plant ‘SUNRENILAPIHO-KAI’ is differ from a similar variety, ‘SUNRENILAPIHO’ in following points,

1. The petal color of ‘SUNRENILAPIHO-KAI’ is pale purplish pink (R.H.S. No. 69D, J.H.S. No. 9501) in upper petal, and vivid purplish red (R.H.S. No. 80A, J.H.S. No. 8906) color and pale purplish pink (R.H.S. No. 69D, J.H.S. No. 9501) center in bottom of bilabiate petal as against those of ‘SUNRENILAPIHO’ is pinkish white (R.H.S.No. 76D, J.H.S.No. 9701), and pinkish white (R.H.S.No. 76D, J.H.S.No. 9701).

2. The diameter of flower of ‘SUNRENILAPIHO-KAI’ is narrower than that of ‘SUNRENILAPIHO’.

3. The peduncle length of ‘SUNRENILAPIHO-KAI’ is shorter than that of ‘SUNRENILAPIHO’.

4. The anthocyanin pigmentation of stem of ‘SUNRENILAPIHO-KAI’ are medium as against these of ‘SUNRENILAPIHO’ are dark.

5. The degree of leaf serration of ‘SUNRENILAPIHO-KAI’ is acute as against that of ‘SUNRENILAPIHO’ is medium.

6. Flowering time of ‘SUNRENILAPIHO-KAI’ is earlier than that of ‘SUNRENILAPIHO’.

7. The degree of heat tolerance of ‘SUNRENILAPIHO-KAI’ is strong as against that of ‘SUNRENILAPIHO’ is medium.

1. The new and distinct variety of Torenia plant, substantially as herein illustrated and described, characterized particularly as to novelty by (A) being a semi-ducumbent and medium branching (B) having large flowers and great profusion of blooms and the whole bush remaining in bloom for considerable period of time, (C) flower petals are pale purplish pink upper petal, vivid purplish red side of bilabiate petal, and vivid purplish red color and pale purplish pink center bottom of bilabiate petal without yellow blotches, (D) having a high resistance to heat, moderate resistance to diseases and pests and weak cold hardiness, (E) growing and having flowers ordinarily in the shade of tree.
